(19) United States
(12) Patent Application Publication
(10) Pub. No.: US 2025/0082806 A1
(43) Pub. Date: Mar. 13, 2025

(54) STERILITY TESTING DRAIN ACTUATOR BASE

(71) Applicant: RAPID MICRO BIOSYSTEMS, INC., Lowell, MA (US)

(72) Inventors: Toby ISAACS, Lowell, MA (US); Ethan FRANCO, Lowell, MA (US); Victor CHUNG, Lowell, MA (US)

(21) Appl. No.: 18/464,066

(22) Filed: Sep. 8, 2023

(51) Int. Cl. A61L 2/28

(52) U.S. Cl. CPC A61L 2/28

(57) ABSTRACT

Exemplary embodiments relate to drain actuators for cassettes used in sterility testing. A sterility cassette may have a spring-loaded drain to allow fluid flow through a porous membrane. To activate the drain, upwards pressure is applied to the spring during kit preparation through an actuator base. The actuator base may automatically actuate the drain when the cassette is inserted into the actuator base, and may be separate from or integral with a thermoformed drain tray. When the sterility cassette is removed from the tray, the base automatically shuts the drain. The actuator base has features that: maintain the rotational alignment between the tray and cassette; actuate the drain port on the bottom of the cassette; minimally inhibit flow out of the drain; minimize the collection of drain fluid; provide a rigid support across the drain tray surface; and stay locked into the drain tray when the cassette is removed.

### BACKGROUND

Sterility testing is a critical process used to determine the absence or presence of viable microorganisms in a product or sample. It is commonly performed on pharmaceuticals, medical devices, and other sterile products to ensure their safety and effectiveness.

Generally speaking, sterility testing may proceed as follows. First, a representative sample of the product or material to be tested may be obtained. A test method may then be selected. Commonly, membrane filtration is the test method applied because of the advantages it provides in terms of sensitivity, versatility, quantification, compatibility, sample recovery, validation, potential for automation, and time efficiency.

In membrane filtration, the sample is filtered through a membrane filter with a defined pore size (typically 0.45 m). The filter retains any microorganisms present in the sample, allowing for their subsequent detection. The membrane filter may be provided on a suitable analysis container, such as the sterility cassettes offered by Rapid Micro Biosystems, Inc. of Lowell, Massachusetts. As the sample is filtered through the membrane filter, any remaining sample fluid that passes through the membrane filter may be drained through a drain port in the base of the cassette.

After filtration, the membrane filter is aseptically transferred to an appropriate culture medium that supports the growth of a wide range of microorganisms. The culture medium can be broth or agar-based, depending on the testing method used. The inoculated culture medium is then incubated under suitable conditions, typically at a temperature between 20-40° C., for a specified period, often ranging from 2 to 14 days. This allows any viable microorganisms to grow and form visible colonies.

After the incubation period, the culture media is examined for the presence of microbial growth. The presence of visible colonies indicates a positive result, suggesting the presence of viable microorganisms and thus a failed sterility test. Alternatively, if no visible growth is observed, it indicates a negative result, suggesting the absence of viable microorganisms and a successful sterility test. In the case of positive results, additional tests can be performed to identify the microorganisms present, such as gram staining, biochemical tests, or molecular techniques like polymerase chain reaction (PCR).

The results of the sterility testing, including the test method, sample details, incubation conditions, and results, are documented as part of the testing records.

### BRIEF SUMMARY

Exemplary embodiments provide a sterility cassette having a spring-loaded drain to allow fluid flow through a porous membrane. To activate the drain, upwards pressure is applied to the spring during kit preparation through an actuator base. The actuator base may be a plastic component that will automatically actuate the drain when the cassette is inserted into the actuator base, and may be separate from or integral with a thermoformed drain tray. When the sterility cassette is removed from the tray, the base automatically shuts the drain due to action of the spring. The actuator base may be injection molded out of a rigid thermoplastic (e.g., polycarbonate) that can withstand ethylene oxide (ETO) sterilization.

The actuator base is designed to interface between the drain tray and the drain in the sterility cassette. The actuator base has features that: maintain the rotational alignment between the tray and cassette; actuate the drain port on the bottom of the cassette; minimally inhibit flow out of the drain; minimize the collection of drain fluid; provide a rigid support across the drain tray surface; and stay locked into the drain tray when the cassette is removed.

In one aspect, a drain actuator for a sterility cassette includes a drain actuator base having a drain outlet defined therein, and a drain actuator provided in the drain outlet and configured to interface with a drain plunger base of a drain plunger provided in the sterility cassette, the drain actuator sized and shaped to provide an opening pressure on the drain plunger when the drain plunger base interfaces with the drain actuator.

The drain actuator may be substantially triangular in shape. In another embodiment, the drain actuator has a flat surface for interfacing with the drain plunger base, the flat surface provided between rounded sides.

The drain actuator may also include include one or more alignment features sized and shaped to mate with corresponding features of a drain tray to maintain a rotational alignment between the drain tray and the sterility cassette.

The drain actuator base may include one or more ridges that extend in a radial direction across a surface of the drain actuator base. The drain actuator may include an inner circumferential ridge around the drain outlet. The drain actuator may include an outer circumferential ridge provided at an outer circumference of the drain actuator base.

The drain actuator may also include one or more fasteners configured to fix the drain actuator to a drain tray.

The drain actuator may be formed from a thermoplastic, such as a rigid thermoplastic from which the drain actuator is molded. The thermoplastic may be selected from the group of thermoplastics capable of withstanding a sterilization process to be applied to the sterility cassette, such as ethylene oxide (ETO) sterilization.

In another embodiment, a sterility testing kit includes a sterility cassette includes a drain assembly, and the drain actuator of claim 1. The drain assembly may include a spring, a drain plunger, and/or a sealing element. The kit may further include a drain tray.

In another embodiment, a method of assembling a sterility kit includes inserting a sterility cassette onto an actuator base, the inserting causing a drain in the sterility cassette to automatically open, injecting a sample into the sterility cassette, the sample being filtered through a membrane, and removing the sterility cassette from the actuator base, the removing causing the drain in the sterility cassette to automatically close.

The method may also include subjecting the drain actuator to sterilization, such as ethylene oxide sterilization, vaporized hydrogen peroxide (VHP) sterilization, chlorine dioxide (ClO2) sterilization, nitrogen dioxide (NO2) sterilization, x-ray sterilization, gamma sterilization, etc.

The method may also include assembling the drain on a base assembly of the sterility cassette.

The actuator base may be integral with a drain tray, or may be attached to the drain tray. Removing the sterility cassette from the actuator base may cause the actuator base to remain attached to the drain tray.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary cassette assembly in accordance with one embodiment.

FIG. 2 is a close-up of a portion of a cross-sectional view of the cassette assembly in accordance with one embodiment.

FIG. 3 depicts an exemplary base assembly for a sterility cassette including a drain assembly in accordance with one embodiment.

FIG. 4 is a close-up of a portion of a cross sectional view of the base assembly in accordance with one embodiment.

FIG. 5A is a cross-sectional view of an actuator base interfacing with the base assembly and the drain in an open position in accordance with one embodiment.

FIG. 5B is a perspective view of a base assembly in accordance with one embodiment.

FIG. 5C is a cross-sectional view of an actuator base interfacing with the base assembly and the drain in a closed position in accordance with one embodiment.

FIG. 6 is a perspective view of an alternative base assembly design in accordance with one embodiment.

FIG. 7 depicts an exemplary base assembly affixed to a drain tray in accordance with one embodiment.

FIG. 8 depicts an assembled cassette attached to the base assembly and drain tray of FIG. 7, in accordance with one embodiment.

FIG. 9 is a flowchart depicting an exemplary procedure for using a drain actuator with a sterility testing kit in accordance with one embodiment.

### DETAILED DESCRIPTION

As noted above, during the filtration process the remaining sample fluid may be drained through a drain port in the base of the cassette. Conventional systems often employ a manual drain port, such as a plug or flip-out drain. Because such drains require manual activation, they increase the labor required to perform sterility testing, decrease the possibilities for automation, and increase the time required to perform an analysis.

Conventional drains also suffer from problems of back pressure. In the context of filtration processes, back pressure is often encountered when a fluid is passed through a filter medium. As the fluid flows through the filter, the presence of the filter medium, particularly if it is fine or densely packed, creates resistance to the flow. This resistance generates a pressure that opposes the flow.

High back pressure in filtration systems can have several undesirable effects. It can reduce the flow rate of the fluid through the filter, impacting the overall filtration efficiency and throughput. Additionally, high back pressure can increase the pressure drop across the system and impact the integrity or lifespan of the filter medium itself. Thus, managing back pressure is crucial in fluid systems to ensure optimal operation and prevent any adverse effects.

Furthermore, conventional drains may experience issues with fluid retention. When fluid is retained in the cassette after filtration, it can lead to inaccurate results. Due to the shape and configuration of conventional drain ports, it can be very difficult to effectively remove all of the fluid that has filtered through the membrane from the bottom of the cassette.

Accordingly, an improved drain system for cassettes would be beneficial to improve throughput and accuracy in sterility testing.

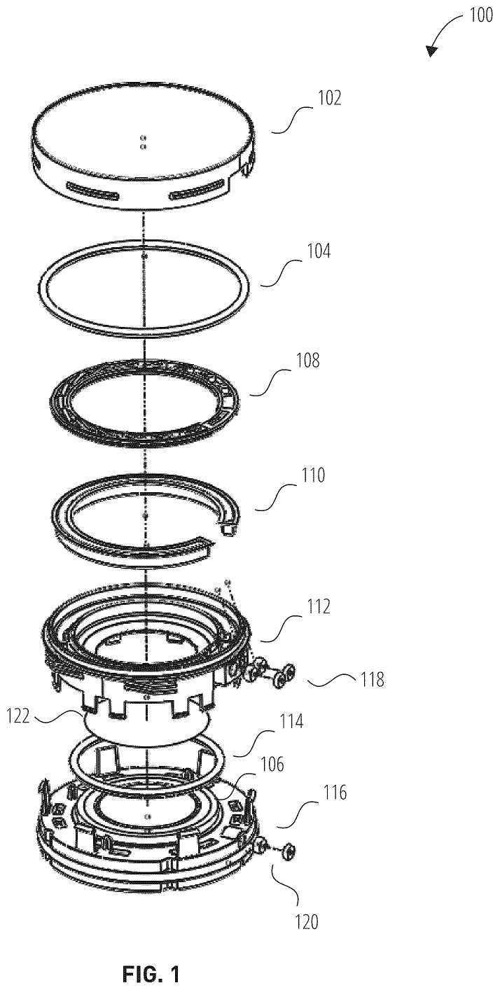

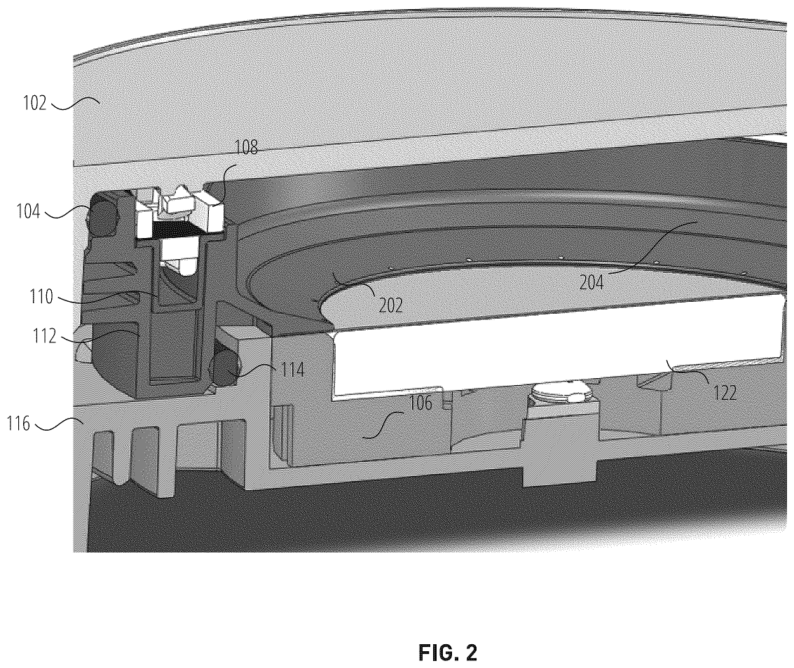

An example of a cassette assembly 100 is shown in FIG. 1, and a cross-sectional side-view is shown in FIG. 2. The cassette assembly 100 may provide a sterile environment for testing. The cassette assembly 100 may provide an anaerobic or aerobic environment, depending on the application.

From top to bottom in FIG. 1, the exemplary cassette assembly 100 includes a lid 102, an o-ring 104, an optional foil cutter 108, a scavenging tray assembly 110, a mid-body assembly 112, a membrane filter 122, a second o-ring 114, and a base assembly 116.

The base assembly 116 forms the bottom-most part of the cassette assembly 100 and serves as a supporting structure to which the other parts can be mounted. The base assembly 116 may be sized and shaped so as to be accommodated in an appropriate testing or analysis device.

A membrane filter 122 may be provided on the base assembly, between the base assembly 116 and the mid-body assembly 112. The membrane filter 122 may be a part of a media pad sized and shaped to be accommodated by a corresponding recess in the base assembly 116. The membrane filter 122 may be any suitable filter, and may have characteristics (such as a desired porosity) selected based on the particular application (e.g., the size of the microorganisms of interest that are intended to be captured by the membrane filter 122). In some embodiments, more than one membrane filter 122 may be provided, which may include multiple different types of membrane filters 122.

Target fluids for analysis may be passed through the membrane filter 122 and into the base assembly 116. The base assembly 116 may include a drain port 120 that allows the fluids to be removed from the cassette assembly 100 after filtration. The drain port 120 may include an opening provided in a part of the base assembly 116 internal to the cassette assembly 100 that connects to a specially shaped outlet on the exterior side of the cassette assembly 100. The outlet may be sized and shaped to mate with a drain manifold that receives the removed fluid and delivers it to an appropriate disposal location.

An o-ring 114 may be provided between the base assembly 116 and the mid-body assembly 112 to prevent fluid from leaking around and therefore bypassing the membrane filter 122. The mid-body assembly 112 includes a mid-body inlet 118 that allows the target fluid (or fluids) being analyzed to be admitted into the cassette assembly 100. The mid-body inlet 118 may include an opening provided in a part of the mid-body assembly 112 internal to the cassette assembly 100 that connects to an opening on the exterior side of the cassette assembly 100. Within the mid-body inlet 118 may be a structure, such as a rubber septum, that seals the cassette assembly 100. To admit a target fluid into the cassette assembly 100, a needle may be used to pierce the structure in the mid-body inlet 118 and deliver the fluid at a relatively high pressure.

In some embodiments, more than one mid-body inlet 118 may be included in the mid-body assembly 112. For example, one mid-body inlet 118 may be provided for admitting a first sample (target fluid of interest for analysis) into the cassette assembly 100, while a second mid-body inlet 118 is provided for admitting a second, different sample. In other embodiments, a first mid-body inlet 118 may be provided for admitting a sample, while a second mid-body inlet 118 may be provided for admitting a growth medium.

The top of the mid-body assembly 112 may be shaped to accommodate a scavenging tray assembly 110, which may include a scavenging material that (for example) absorbs oxygen in the cassette assembly 100. The scavenging tray assembly 110 may be topped by foil that holds the scavenging material in place and protects it from outside air until the scavenging tray assembly 110 is deployed in the cassette assembly 100. To release the scavenging material, the cassette assembly 100 may be provided with a foil cutter 108 designed to penetrate the foil and allow the scavenging material to scavenge the environment within the sealed cassette assembly 100.

To seal the cassette assembly 100, an o-ring 104 may be placed on top of the mid-body assembly 112, and then a lid 102 may be used to cap the entire assembly. As shown in FIG. 2, the o-ring 104 forms a seal between the mid-body assembly 112 and the lid 102 and prevents the fluid from leaking from the top of the cassette assembly 100 (and seals the interior of the cassette assembly 100 to allow the scavenging material to scavenge the environment of oxygen).

As further shown in FIG. 2, the mid-body assembly 112 may include a mid-body assembly floor 202 that extends from an inner circumferential wall 204 of the mid-body assembly 112 towards an interior of the cassette assembly 100 in the radial direction. The mid-body assembly floor 202 may be slanted towards the membrane filter 122 to encourage the fluid to flow towards the membrane filter 122.

Although exemplary embodiments are described with reference to the depicted cassette assembly configuration for purposes of illustration, one of skill in the art will recognize that other types of cassette assemblies (with more, fewer, or a different configuration of parts) or other sterile environments may also be used. Moreover, although exemplary embodiments are described in terms of sterility testing using membrane filtration (and the structure in FIG. 1 and FIG. 2 is configured accordingly), other applications of the splashguard described below will be readily apparent.

Note that FIG. 1 and FIG. 2 depict the drain port 120 on the side of the base assembly 116. Although such a configuration can be used with the embodiments described herein, the embodiments described below instead move the drain port 120 to the bottom center of the base assembly 116, which may allow for better draining.

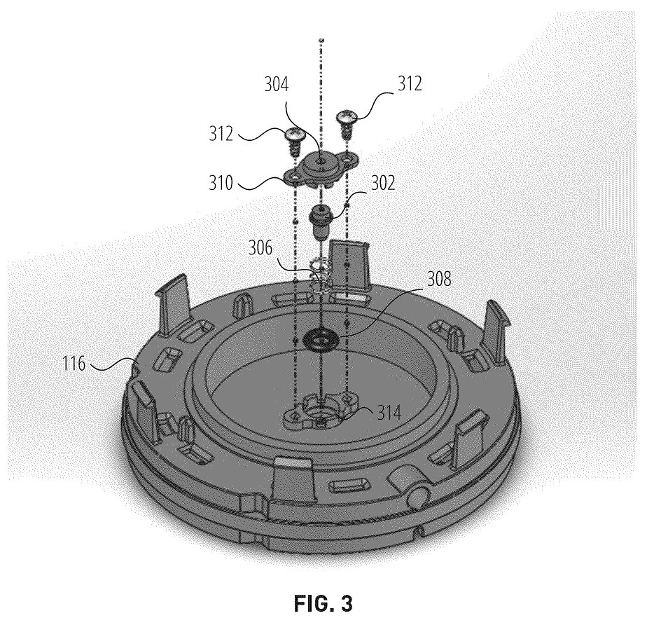

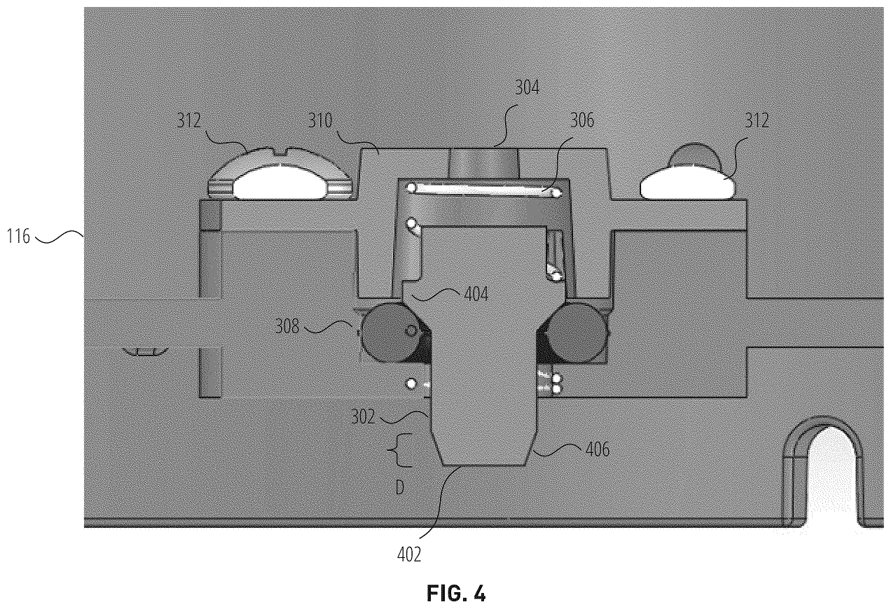

For example, FIG. 3 depicts an exemplary base assembly 116 having a drain assembly deployed on it. The drain assembly includes a drain plunger 302, a spring 306, a sealing element 308, a drain support 310, and one or more fasteners 312. These elements are depicted in cross section in FIG. 4.

Note that in the description of these and following figures, directions such as “upwards” and “downwards” are referenced. Generally, “downwards” corresponds to the direction of gravity, since fluids will generally drain in this direction. However, in some circumstances it may be possible that fluids will flow out of the drain port in a direction other than the gravitational direction; in these circumstances, the direction in which the fluid drains may be considered the “downward” direction and the opposite direction may be considered “upwards.” Further directions include a longitudinal direction, which refers to a direction extending along an axis that extends through a center of the base assembly 116 in the direction in which the sterility cassette is assembled. A radial direction extends outwards from this central axis along a radius from the center point.

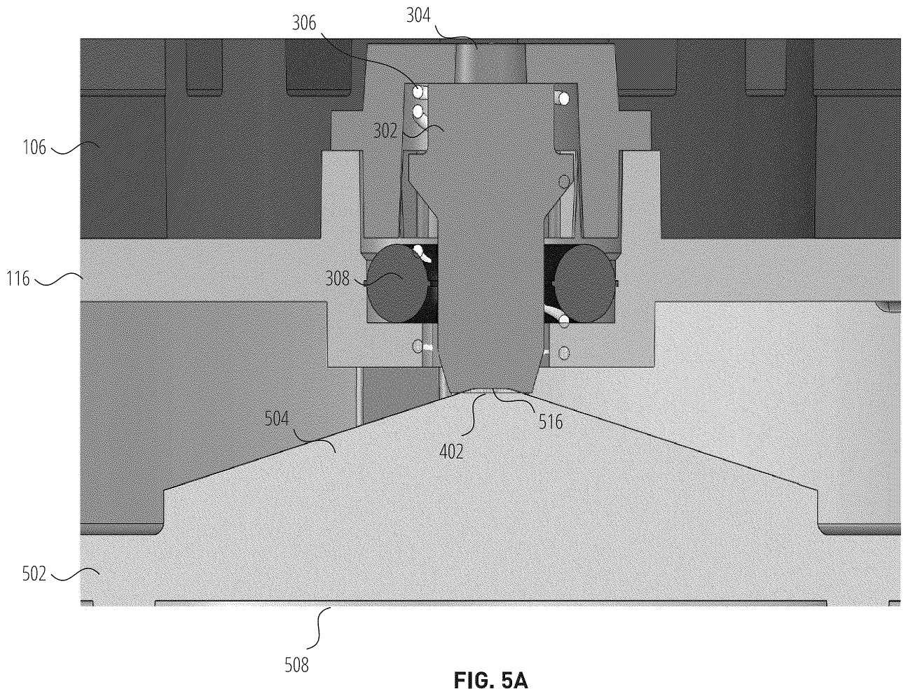

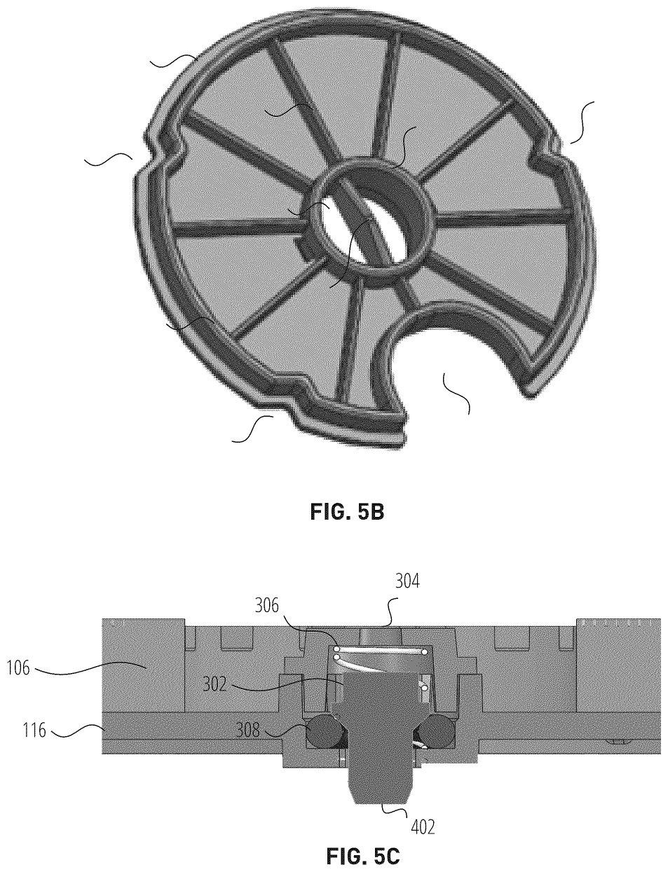

The drain plunger 302 is a component configured to fit over and/or extend into a base assembly drain port 314 to prevent fluid from flowing through the base assembly drain port 314 when the drain plunger 302 is in a closed position. The bottom side of the drain plunger 302 forms a drain plunger base 402 configured to interface with a drain actuator 504, as described in more detail in connection with FIG. 5A.

The drain plunger 302 includes a circumferential drain plunger flange 404. The drain plunger flange 404 is configured and positioned on the drain plunger 302 so as to interface with the sealing element 308. When in a closed position, the drain plunger flange 404 presses against the sealing element 308, thereby sealing the base assembly 116 and preventing fluid from flowing out of a base assembly drain port 314.

Optionally, the drain plunger 302 may include tapered sides 406 in the vicinity of the drain plunger base 402. Because the sides of the drain plunger 302 are tapered in this region, they are narrower than the rest of the body of the drain plunger 302. Accordingly, when the drain plunger 302 is lifted upwards by a certain distance d (which may substantially correspond to the length of the tapered side 406), the tapered sides 406 create a gap between the wall of the base assembly 116 and the drain plunger 302. The fluid being drained can then pass between the drain plunger flange 404 and the sealing element 308, and from there past the tapered sides 406 and out the base assembly drain port 314. Alternatively or in addition, the tapered sides 406 may serve to guide the drain plunger 302 back into the base assembly drain port 314 when it closes.

The spring 306 biases the drain plunger 302 into a closed position. In order to open the drain, upwards pressure may be applied to the drain plunger base 402 to push the drain plunger 302 against the spring 306, causing the plunger to withdraw from an opening in the base assembly 116 that forms the base assembly drain port 314.

The sealing element 308 may be any element capable of sealing the base assembly drain port 314 against fluid intrusion. For example, the sealing element 308 may be an o-ring.

The drain support 310 is a component configured to support the drain plunger 302 hold the drain plunger 302 and spring 306 against the base assembly 116. The drain support 310 is provided with drain opening 304 through which fluid such as a filtered sample may pass. The drain support 310 may further include openings for fasteners 312, which allow the drain support 310 to be secured to the base assembly 116.

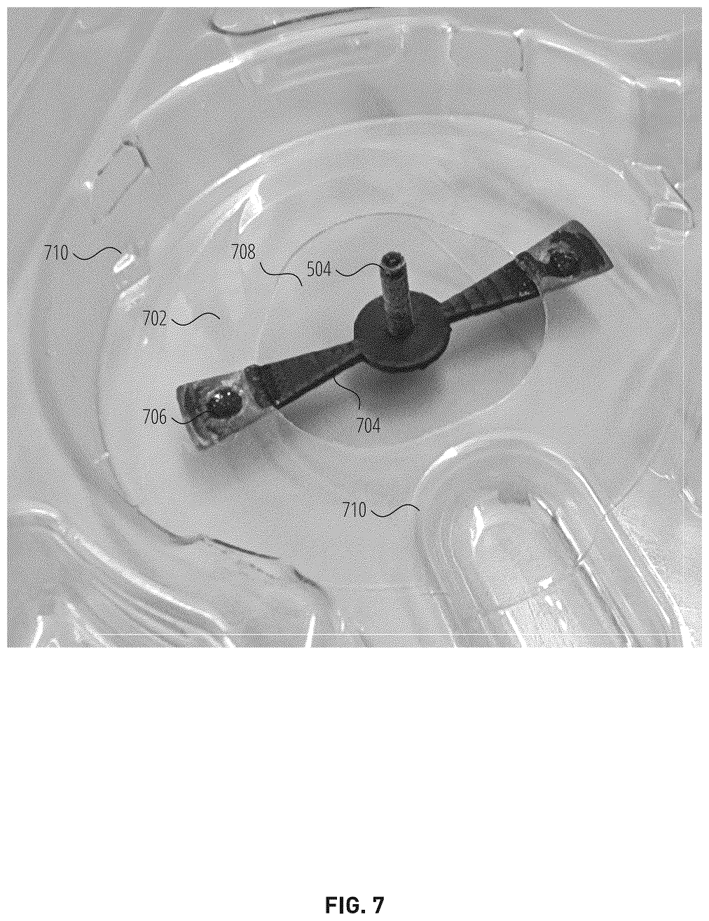

Turning to FIG. 5A-FIG. 5C, the base assembly 116 may be deployed onto an actuator base 502. In practice, the actuator base 502 may be a part of, or may be attached to, a drain tray (the drain tray is described in more detail in connection with FIG. 7). The drain tray and/or actuator base 502 may include interlocking features that allow the drain tray and actuator base 502 to be temporarily locked together when the actuator base 502 is placed on top of the drain tray. When the base assembly 116 is connected to the actuator base 502, a drain actuator 504 on the actuator base 502 presses against the drain plunger base 402 of the drain plunger 302, which causes the drain plunger 302 to press against the spring 306 and open. This occurs automatically when the base assembly 116 is pressed onto the actuator base 502.

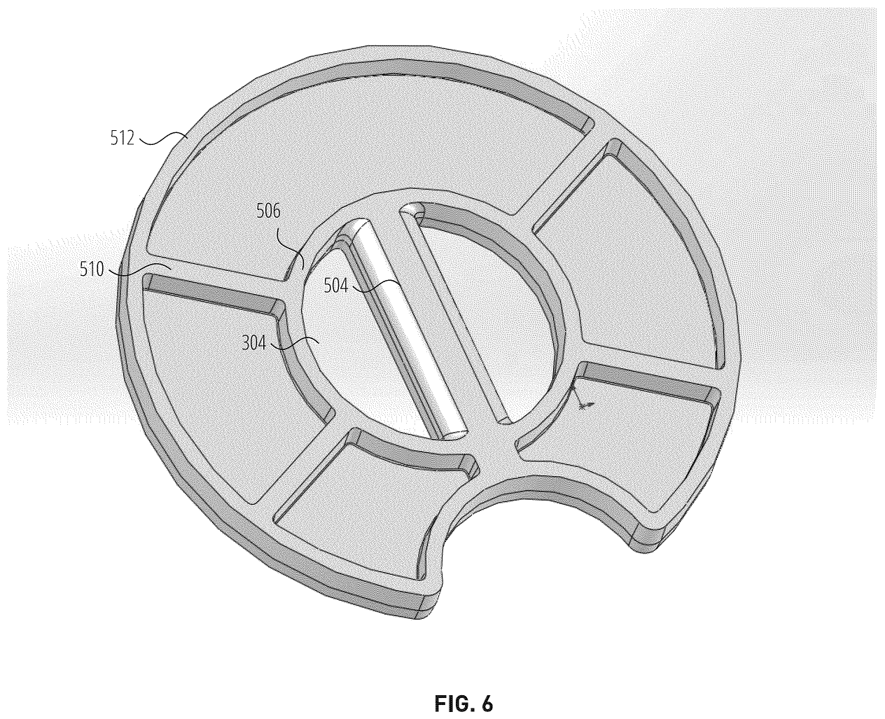

In this example, the drain actuator 504 is substantially triangular in shape. The sides of the drain actuator 504 may have one or more slopes; in this example, the drain actuator 504 slopes up to a flattened top surface 516 which makes contact with the drain plunger base 402. Due to the sloped sides, only the top surface 516 contacts the drain plunger base 402, and there is sufficient space between the drain plunger base 402 and the sloped sides of the drain actuator 504 to allow fluid to pass unimpeded out of the drain. The drain actuator 504 could also take on different shapes: for example, it could come to a point or have a rounded top surface 516. For instance, FIG. 6 depicts an example where the drain actuator 504 is a cross-bar having a flat top surface and rounded sides. The flat top surface engages with the drain plunger base 402 and pushes it upwards, while the rounded sides provide a standoff area that retreats away from the base assembly 116, thereby allowing fluid to flow out of the drain.

Returning to FIG. 5A, the base assembly 116 rests on an inner circumferential ridge 506 of the actuator base 502. Within the inner circumferential ridge 506 and passing through the actuator base 502 is a drain outlet 508 that allows the fluid to pass from the base assembly 116 out past the actuator base 502 and into a drain tray.

The shapes of the drain actuator 504 in FIG. 5A and FIG. 6 allow the drain to be actuated with minimal contact between the actuator base 502 and the base assembly 116. Because of this, fluid flow is less obstructed than it might otherwise be, which allows for improved drainage (thus reducing back pressure and fluid retention in the base assembly 116).

In addition to actuating the drain plunger 302, the drain actuator 504 acts as a rigid support, which is particularly useful when the actuator base 502 is relatively thin and flexible (or when the actuator base 502 is a part of a drain tray, which are conventionally flimsy structures). This provides an additional advantage in lending the actuator base 502 (and/or drain tray) rigidity and preventing it from buckling when the base assembly 116 is pressed into the drain actuator 504.

As shown in FIG. 5B and FIG. 6, the actuator base 502 includes one or more ridges 510, which also support the stability and rigidity of the actuator base 502. The base assembly 116 may also rest on an outer circumferential ridge 512, providing additional contact points between the actuator base 502 and the base assembly 116 away from the area of the drain, so that flow through the drain is not obstructed. The outer circumferential ridge 512 also serves to strengthen the actuator base 502 and lend it additional stability and rigidity.

Furthermore, the actuator base 502 may be provided with one or more alignment features 514. These cutouts in the shape of the actuator base 502 at its outer circumference are sized and configured to mate with corresponding features in the drain tray (see, e.g., FIG. 7). In this manner, the actuator base 502 (and, consequently, the base assembly 116) can be prevented from rotating as pressure is applied to the drain plunger 302. This allows for rotational alignment between the sterility cassette and the drain tray to be maintained, which may be important when attempting to line up the injection port of the sterility cassette so that the sample can be introduced to the cassette.

FIG. 5A depicts the drain plunger 302 in a closed position with the base assembly 116 positioned on top of the actuator base 502. When the base assembly 116 is removed from the actuator base 502, as shown in FIG. 5C, the spring 306 pushes against the drain plunger 302, which closes the drain outlet 508.

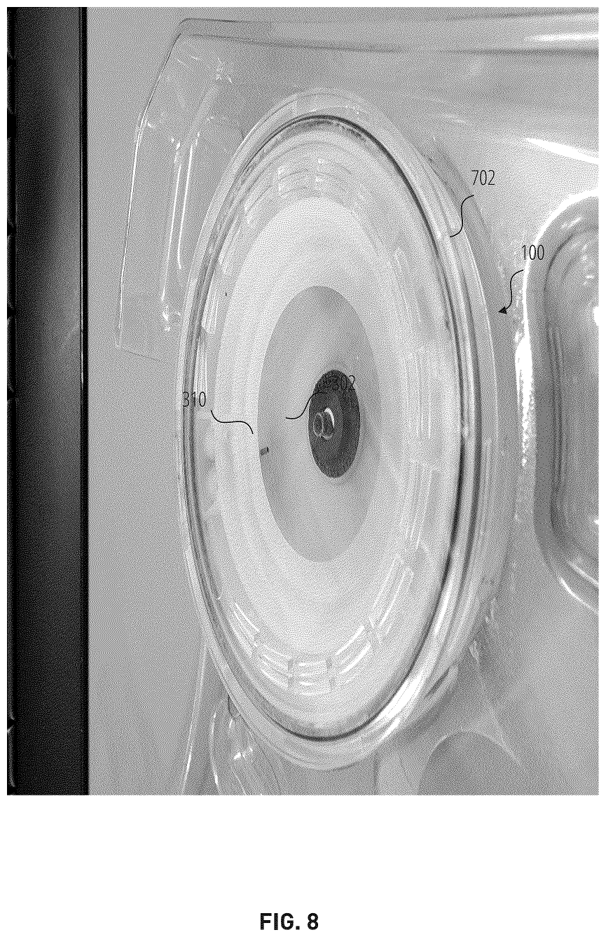

FIG. 7 and FIG. 8 depict an exemplary drain tray 702 with an alternative actuator design affixed to it.

In sterility testing, a drain tray 702 refers to a specially designed tray or container used to collect and contain the excess sterilizing agent or media during the testing process. Because sterility testing is conducted to determine the presence or absence of viable microorganisms in a sample or product, the drain tray 702 helps prevent cross-contamination and facilitates the safe disposal of the sterilizing agent or media after the testing is complete.

During sterility testing, the test samples are often placed in containers such as vials, ampoules, syringes, or the above-described sterility cassette. These containers may be filled with a sterilizing agent or growth media to support microbial growth in case any microorganisms are present in the sample. The excess sterilizing agent or media that is not absorbed by the test sample needs to be drained away to prevent dilution or interference with the test results.

The drain tray 702 is typically placed underneath the test samples to collect any excess liquid, ensuring it doesn't come into contact with other samples or contaminate the testing area. It may have an inclined or perforated surface to facilitate drainage and prevent pooling of liquid. The tray is often made of materials that are compatible with the sterilizing agent or media used in the testing process.

Once the testing is complete, the drain tray 702 can be easily removed, and the sterilizing agent or media can be disposed of according to established protocols and safety guidelines. The use of a drain tray 702 helps to maintain the integrity and accuracy of the test results while ensuring proper containment and disposal of potentially contaminated materials.

In this example, the actuator is represented by a relatively simple design having a drain actuator 504 in the form of a post with a tapered top connected to two actuator support arms 704. The drain actuator 504 engages with the drain plunger 302 as previously discussed. The actuator support arms 704 serve as the rigid support for the drain actuator 504 and connect via actuator fasteners 706 to the actuator support arm 704. The actuator fastener 706 may include, for example, screws, mating tabs, adhesives such as glue, etc. Alternatively or in addition, the actuator base 502 including the drain actuator 504 and the actuator support arm 704 may simply be integral with the 704.

A drain tray opening 708 allows fluid to pass through the drain tray 702 in this example, potentially into a secondary collection area. The drain tray opening 708 may be optional such that the fluid instead passes into the drain tray 702 itself, from which it can be discarded or reused and the drain tray 702 can be cleaned.

As previously noted, the drain tray 702 includes tray alignment features 710 that correspond to the size, shape, and location of the alignment features 514 on the actuator base 502. These mating features allow the actuator base 502 and/or base assembly 116 to be secured against rotation while the drain actuator 504 is engaged with the drain plunger 302.

Although FIG. 7 is depicted with a particular actuator design, certain features from FIG. 7 are generalizable to other actuator designs. For example, the actuator fasteners 706 may be provided on the actuator base 502 shown in FIG. 5B to allow the actuator base 502 to be fixed to the drain tray 702. The actuator fasteners 706 may be provided on the top of the actuator base 502 (so that the actuator base 502 fastens to the drain tray 702 from underneath) or on the bottom of the actuator base 502 (so that the actuator base 502 fastens to the drain tray 702 from the top).

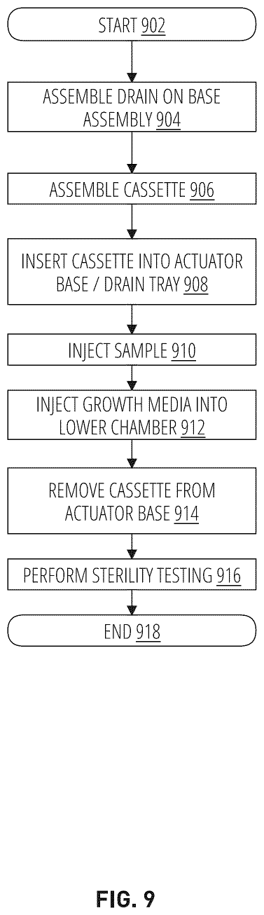

FIG. 9 illustrates an example method for using an actuator base 502 with a sterility cassette as part of a sterility kit. Although the example method depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

Some or all of the steps described below may be performed automatically by a suitable sterility kit assembly apparatus, such as a robotic system capable of manipulating the sterility cassette and automatically introducing the sample fluid to the sterility cassette.

The method may begin at block 902, when a sterility testing process is begun. At block 904, a sterility cassette including a base assembly 116 may be accessed and a drain may be assembled on the base assembly 116. In some embodiments, the base assembly 116 may already have a drain assembly installed prior to beginning the sterility process (e.g., it might have been installed by the manufacturer or a third party, or might be integral with the base assembly 116). In one example, a user may place the sealing element 308 into the base assembly drain port 314, and then place the spring 306 and drain plunger 302 into the central opening of the sealing element 308. The drain support 310 may be lowered onto the spring 306 and drain plunger 302, and the fasteners 312 may be applied to secure the drain support 310 to the base assembly 116.

At block 906, the remainder of the sterility cassette may be assembled. This may involve (depending on the specific structure of the particular cassette used) placing the membrane support 106 on the base assembly 116, adding an appropriate membrane filter 122 on top of the membrane support 106, snapping the mid-body assembly 112 into the base assembly 116 over the membrane filter 122 and membrane support 106, optionally deploying a scavenging tray assembly 110 and/or foil cutter 108 in appropriate recesses in the mid-body assembly 112, adding the o-ring 104 to the mid-body assembly 112, and topping the assembly with the lid 102.

At block 908, the assembled cassette may be inserted into a drain tray 702. The drain tray may include an integrated actuator base 502, or an actuator base 502 may optionally be installed on or attached to the drain tray 702 before inserting the assembled cassette. When the cassette is inserted onto the drain tray, the drain actuator 504 makes contact with the drain plunger base 402 and pushes against the spring 306, causing the drain plunger 302 to open automatically.

With the drain plunger open, at block 910 the sample may be injected (e.g., through the mid-body inlet 118. The sample may be filtered through the membrane filter 122 and any remaining fluid not retained by the membrane filter 122 may be passed to the bottom of the base assembly 116, through the drain opening 304, around the drain plunger 302, and out through the drain opening 304 and drain outlet 508 to the drain tray (or a secondary collecting area).

At block 912, growth media may be injected into a lower chamber of the base assembly 116 (e.g., below the membrane filter 122). Any excess growth media may be removed through the drain outlet 508 in a manner similar to the sample fluid.

When the sample has been filtered through the membrane and any other desired fluids (e.g., a rinse solution) have been filtered and drained, at block 914 the cassette may be removed from the drain tray 702 (and, correspondingly, the actuator base 502). When the cassette is removed, the drain plunger base 402 is withdrawn from the drain actuator 504 until the drain actuator 504 no longer makes contact with the drain plunger base 402. Under action of the spring 306, the drain plunger 302 automatically closes and seals the inside of the sterility cassette.

Sterility testing may then be performed on the sealed cassette at block 916, after which the method ends at done block 918.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

Some embodiments may be described using the expression “coupled” and “connected” along with their derivatives. These terms may indicate that two or more elements are in direct physical or electrical contact with each other. However, they may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A drain actuator for a sterility cassette comprising:
a drain actuator base having a drain outlet defined therein; and
a drain actuator provided in the drain outlet and configured to interface with a drain plunger base of a drain plunger provided in the sterility cassette, the drain actuator sized and shaped to provide an opening pressure on the drain plunger when the drain plunger base interfaces with the drain actuator.

2. The drain actuator of claim 1, wherein the drain actuator is substantially triangular in shape.

3. The drain actuator of claim 1, wherein the drain actuator has a flat surface for interfacing with the drain plunger base, the flat surface provided between rounded sides.

4. The drain actuator of claim 1, further comprising one or more alignment features sized and shaped to mate with corresponding features of a drain tray to maintain a rotational alignment between the drain tray and the sterility cassette.

5. The drain actuator of claim 1, wherein the drain actuator base comprises one or more ridges that extend in a radial direction across a surface of the drain actuator base.

6. The drain actuator of claim 1, further comprising an inner circumferential ridge around the drain outlet.

7. The drain actuator of claim 1, further comprising an outer circumferential ridge provided at an outer circumference of the drain actuator base.

8. The drain actuator of claim 1, further comprising one or more fasteners configured to fix the drain actuator to a drain tray.

9. The drain actuator of claim 1, wherein the drain actuator is formed from a thermoplastic.

10. A sterility testing kit comprising:
a sterility cassette comprising a drain assembly; and
the drain actuator of claim 1.

11. The kit of claim 10, wherein the drain assembly comprises a spring.

12. The kit of claim 10, wherein the drain assembly comprises the drain plunger.

13. The kit of claim 10, wherein the drain assembly further comprises a sealing element.

14. The kit of claim 10, further comprising a drain tray.

15. A method of assembling a sterility kit comprising:
inserting a sterility cassette onto an actuator base, the inserting causing a drain in the sterility cassette to automatically open;
injecting a sample into the sterility cassette, the sample being filtered through a membrane; and
removing the sterility cassette from the actuator base, the removing causing the drain in the sterility cassette to automatically close.

16. The method of claim 15, further comprising subjecting the drain actuator to sterilization.

17. The method of claim 15, further comprising assembling the drain on a base assembly of the sterility cassette.

18. The method of claim 15, wherein the actuator base is integral with a drain tray.

19. The method of claim 15, further comprising attaching the actuator base to a drain tray.

20. The method of claim 19, wherein removing the sterility cassette from the actuator base causes the actuator base to remain attached to the drain tray.
